(19) United States
(12) Patent Application Publication
(10) Pub. No.: US 2023/0312669 A1
(43) Pub. Date: Oct. 5, 2023

(54) VIRAL RECEPTOR-DERIVED PEPTIDES AGAINST VIRAL DISEASES

(71) Applicant: Howard University, Washington, DC (US)

(72) Inventors: Shaolei Teng, Washington, DC (US); Qiyi Tang, Washington, DC (US)

(73) Assignee: Howard University, Washington, DC (US)

(21) Appl. No.: 18/173,264

(22) Filed: Feb. 23, 2023

Related U.S. Application Data
(60) Provisional application No. 63/313,477, filed on Feb. 24, 2022.

(51) Int. Cl. C07K 14/705; A61P 31/14

(52) U.S. Cl. CPC C07K 14/705; A61P 31/14

(57) ABSTRACT

The present disclosure provides novel computational methods for identifying potential therapeutic peptides as antivirals, and methods of using viral receptor-derived peptides as antivirals. Further, the present disclosure provides methods of using angiotensin converting enzyme 2 (ACE2)-derived peptides as therapeutic treatments for severe acute respiratory syndrome coronavirus 2 (SARS-CoV-2)-caused coronavirus disease 2019 (COVID-19).

### CROSS REFERENCE TO RELATED APPLICATIONS

This application claims benefit from of U.S. Provisional Application No. 63/313,477 filed Feb. 24, 2022, the entire contents of which are hereby incorporated by reference.

### INCORPORATION BY REFERENCE OF SEQUENCE LISTING

The content of the electronically submitted sequence listing, file name: A285316_Sequence Listing.xml; size 6.26 kilobytes, and date of creation: May 23, 2023, is incorporated herein by reference in its entirety.

### FIELD OF THE INVENTION

The present disclosure relates to novel computational methods for identifying novel therapeutic peptides as antivirals. The present disclosure also relates to the production of compositions for treating virus infection, and in particular embodiments, to the production of compositions for treating coronavirus infection, including SARS-CoV-2 infection. The present disclosure further relates to viral receptor-derived peptides as antivirals, as well as ACE2 (angiotensin converting enzyme 2)-derived peptides for applications such as the therapeutic treatment of severe acute respiratory syndrome coronavirus 2 (SARS-CoV-2)-caused coronavirus disease 2019 (COVID-19).

### BACKGROUND INFORMATION

The current pandemic of severe acute respiratory syndrome coronavirus 2 (SARS-CoV-2)-caused coronavirus disease 2019 (COVID-19) has devastating effects. Due to its highly contagious nature with a basic reproduction number (R0) of 2-3, with some strains being even higher, SARS-CoV-2 has proven catastrophic in both prevalence and fatality in the United States. See, e.g., Sanche et al. (“Contagiousness and Rapid Spread of Severe Acute Respiratory Syndrome Coronavirus,” Emerg. Infect. Dis., 2020, 26) and Salzberger et al. (“Epidemiology of SARS-CoV-2”, Infection, 2002, doi:10.1007/s15010-020-01531-3).

Although an accurate final fatality rate can only be obtained when the pandemic is over, existing data suggests that it could be over 10% in some countries. SARS-CoV-2 infects people of all ages, but the elderly and/or people with underlying diseases, such as hypertension, diabetes mellitus, and cardiovascular diseases, are at high risk of experiencing greater disease severity and fatality. See, e.g., Garg et al. (“Hospitalization Rates and Characteristics of Patients Hospitalized with Laboratory-Confirmed Coronavirus Disease 2019—COVID-NET, 14 States,” Morbidity and Mortality Weekly Report, Mar. 1-30, 2020, 69: 1-7); Vuorio et al. (“Familial hypercholesterolemia and COVID-19: triggering of increased sustained cardiovascular risk,”J. Intern. Med., 2020, doi:10.1111/joim.13070); Li et al. (“Clinical characteristics of 25 death cases with COVID-19: a retrospective review of medical records in a single medical center, Wuhan, China,” Int. J. Infect. Dis., 2020, doi:10.1016/j.ijid.2020.03.053); Liu et al. (“The Science Underlying COVID-19: Implications for the Cardiovascular System,” Circulation, 2020, doi:10.1161/CIRCULATIONAHA.120.047549); de Wilde et al. (“Alisporivir inhibits MERS- and SARS-coronavirus replication in cell culture, but not SARS-coronavirus infection in a mouse model,” Virus Res., 2017, 228: 7-13); and Yancy et al. (“COVID-19 and African Americans,” JAMA, 2020, doi:10.1001/jama.2020.6548).

There is a need for novel antivirals which can safely and effectively act against the SARS-CoV-2 infection and combat the associated high prevalence of morbidity and mortality. The development of novel, therapeutic peptides as antivirals may be advantageous, as therapeutic peptides can be safe, highly specific, and tolerable. Antivirals may target and disrupt different aspects of the viral life cycle. This includes attachment to host cells, entry, uncoating, expression and replication of viral genome, assembly, and release of new virus. However, no effective antivirals against SARS-CoV-2 are currently available.

Regarding viral entry mechanisms, the viral spike (S) protein of SARS-CoV-2 is responsible for cell entry. The S protein is cleaved by the human furin enzyme to generate S1, which binds to the host receptor, ACE2 (angiotensin converting enzyme 2), and S2, which promotes viral fusion with the host cell membrane via interaction with transmembrane protease, serine 2 (TMPRSS2), and enables viral entry. See, e.g., Fehr et al. (“Coronaviruses: an overview of their replication and pathogenesis,” Methods Mol. Biol., 2015, 1282: 1-23).

### SUMMARY OF THE INVENTION

The present disclosure addresses the above-described limitations in the art, by providing, amongst others, peptides, compositions and methods relating to the specific targeting of ACE2 binding to prevent SARS-CoV-2 infection, and to preserve physiological functions of ACE2. The present inventors discovered that while peptides which target the S protein can inhibit viral infection, targeting specifically ACE2 binding can prevent SARS-CoV-2 infection and preserve physiological functions of ACE2.

Non-limiting embodiments of the disclosure include as follows:
[1] A composition for the treatment of a viral infection, wherein said composition comprises a first peptide, wherein said first peptide is derived from a wild-type receptor for the virus, and wherein said first peptide is able to bind to said virus.
[2] The composition of [1], wherein said composition is formulated for intranasal administration.
[3] The composition of [1] or [2], wherein said virus is a coronavirus.
[4] The composition of [3], wherein said coronavirus is SARS-CoV-2.
[5] The composition of [4], wherein said first peptide binds to the RBD of a spike protein of the SARS-CoV-2 viral particle.
[6] The composition of [4], wherein said receptor is ACE2.
[7] The composition of [4], wherein said receptor is TMPRSS2.
[8] The composition of [5], wherein said first peptide comprises an amino acid sequence having at least 90% identity to an amino acid sequence selected from the group consisting of SEQ ID NOs: 1-6, and wherein said first peptide is less than 100 amino acids in length.
[9] The composition of [5], wherein said first peptide comprises an amino acid sequence selected from the group consisting of SEQ ID NOs: 1-6, and wherein said first peptide is less than 100 amino acids in length.
[10] The composition of [5], wherein said first peptide consists of an amino acid sequence selected from the group consisting of SEQ ID NOs: 1-6.
[11] The composition of any one of [1]-[7], wherein said first peptide is 10-50 amino acids in length.
[12] The composition of any one of [1]-[11], wherein said composition contains at least one additional peptide derived from a wild-type receptor for the virus, wherein said additional peptide is able to bind to said virus, and wherein said at least one additional peptide has a different amino acid sequence from said first peptide.
[13] The composition of [12], wherein said at least one additional peptide comprises an amino acid sequence having at least 90% identity to an amino acid sequence selected from the group consisting of SEQ ID NOs: 1-6, and wherein said at least one additional peptide is less than 100 amino acids in length.
[14] The composition of [12], wherein said at least one additional peptide comprises an amino acid sequence selected from the group consisting of SEQ ID NOs: 1-6, and wherein said at least one additional peptide is less than 100 amino acids in length.
[15] The composition of [12], wherein said at least one additional peptide consists of an amino acid sequence selected from the group consisting of SEQ ID NOs: 1-6.
[16] A method of treating a viral infection, comprising administering a therapeutically effective amount of the composition of any one of [1]-[15] to a subject in need thereof.
[17] The method of [16], wherein said composition is administered intranasally to a subject in need thereof.
[18] The method of [16] or [17], wherein said first peptide binds to a SARS-CoV-2 viral particle.
[19] The method of [18], wherein said binding inhibits attachment of said SARS-CoV-2 viral particle to an ACE2 receptor on the surface of a cell of said subject.
[20] The method of [19], wherein said administration reduces COVID-19-related morbidity and/or mortality in a patient infected with SARS-CoV-2.
[21] A method of producing a viral receptor-derived peptide, said method comprising quantifying an effect of a coding mutation on receptor stability and/or receptor-virus binding affinity, wherein said peptide is derived from a region of said viral receptor that includes the residue subject to said coding mutation.
[22] The method of [21], wherein the effect of said coding mutation is quantified using computational mutagenesis.
[23] The method of [21], wherein the peptide is chemically synthesized.
[24] The method of [21], wherein the peptide is produced by recombinant protein expression.
[25] The method of [21], wherein said viral receptor is a human protein.
[26] The method of [21], wherein a binding energy change is used to determine the effect of said coding mutation on receptor stability and/or receptor-virus binding affinity.
[27] A method for the treatment of a coronavirus infection in a patient who has an underlying comorbidity consisting of being 65 years or older, having hypertension, having diabetes mellitus, and/or having cardiovascular disease, wherein said method comprises administering a therapeutically effective amount of the composition of any one of [4]-[10] and [13]-[15] to a subject in need thereof.
[28] The composition of [12], wherein said first peptide is an ACE2-derived peptide, and wherein said at least one additional peptide is a TMPRSS2-derived peptide.
[29] A method of treating a viral infection, comprising administering a therapeutically effective amount of the composition of [28] to a subject in need thereof, and wherein said administration has an additive or synergistic effect on treating the viral infection.

### INCORPORATION BY REFERENCE

All patents, publications, and patent applications cited in the present specification are herein incorporated by reference as if each individual patent, publication, or patent application was specifically and individually indicated to be incorporated by reference in its entirety for all purposes.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 depicts the role of SARS-CoV-2 and its S protein in aggravating underlying diseases.

FIG. 2 depicts a structural representation of (A, B) SARS-CoV-2 S (RBD: yellow orange; RBM: orange), human ACE2 (green; interacting region: cyan), and (C) human TMPRSS2.

FIG. 3 depicts the contribution of (A) the stability effects of ACE2 mutations, and (B) the binding affinity effects of ACE2.

FIG. 4 depicts the effects of residues and mutations on RBD-ACE2 interaction. The line chart summarizes the binding energy changes for ΔΔΔG mean of residues (bar) and ΔΔΔG of substitutions to Alanine (circle) in ACE2 residues. The heatmap show the ΔΔΔG of all ACE2 mutations.

FIG. 5 depicts mutation pathogenicity analysis. (A) Pie charts summarize the contribution of neutral (blue) and damaging (red) mutations in human ACE2, and (B) depicts a heatmap of mutation pathogenicity for human ACE2.

FIG. 6 depicts the effects of ACE2-derived peptides (SEQ ID NOs: 1-6, respectively, from top to bottom) on SARS-CoV-2 infection, using a Pseudo-typed SARS-CoV-2 viral system on BHK21-hACE2 cell lines.

### DETAILED DESCRIPTION OF THE INVENTION

As discussed herein, the present disclosure provides, amongst others, peptides, compositions and methods relating to the specific targeting of ACE2 binding to prevent SARS-CoV-2 infection, and to preserve physiological functions of ACE2. The present inventors discovered that while peptides which target the S protein can inhibit viral infection, targeting specifically ACE2 binding can prevent SARS-CoV-2 infection and preserve physiological functions of ACE2. Preserving the physiological functions of ACE2 may alter the mechanism underlying the increased risk of fatality of COVID-19 in patients with high-risk underlying diseases. This includes individuals with hypertension, diabetes mellitus, and cardiovascular diseases.

It is to be understood that the terminology used herein is for the purpose of describing particular embodiments only and is not intended to be limiting. As used in the present specification and the appended claims, the singular forms “a,” “an,” and “the” include plural referents unless the context clearly dictates otherwise. Thus, for example, reference to “a polynucleotide” includes one or more polynucleotides, and reference to “a vector” includes one or more vectors.

Unless defined otherwise, all technical and scientific terms used herein have the same meaning as commonly understood by one of ordinary skill in the art to which the invention pertains. Although other methods and materials similar, or equivalent, to those described herein can be useful in the present invention, preferred materials and methods are described herein.

In view of the teachings of the present specification, one of ordinary skill in the art can apply conventional techniques of immunology, biochemistry, chemistry, molecular biology, microbiology, cell biology, genomics, and recombinant polynucleotides, as taught, for example, by the following standard texts: Abbas et al. (Cellular and Molecular Immunology, 2017, 9th Edition, Elsevier, ISBN 978-0323479783); Butterfield et al. (Cancer Immunotherapy Principles and Practice, 2017, 1st Edition, Demos Medical, ISBN 978-1620700976); Kenneth Murphy (Janeway's Immunobiology, 2016, 9th Edition, Garland Science, ISBN 978-0815345053); Stevens et al. (Clinical Immunology and Serology: A Laboratory Perspective, 2016, 4th Edition, Davis Company, ISBN 978-0803644663); E. A. Greenfield (Antibodies: A Laboratory Manual, 2014, Second edition, Cold Spring Harbor Laboratory Press, ISBN 978-1-936113-81-1); R. I. Freshney (Culture of Animal Cells: A Manual of Basic Technique and Specialized Applications, 2016, 7th Edition, Wiley-Blackwell, ISBN 978-1118873656); C. A. Pinkert (Transgenic Animal Technology, Third Edition: A Laboratory Handbook, 2014, Elsevier, ISBN 978-0124104907); H. Hedrich (The Laboratory Mouse, 2012, Second Edition, Academic Press, ISBN 978-0123820082); Behringer et al. (Manipulating the Mouse Embryo: A Laboratory Manual, 2013, Fourth Edition, Cold Spring Harbor Laboratory Press, ISBN 978-1936113019); McPherson et al. (PCR 2: A Practical Approach, 1995, IRL Press, ISBN 978-0199634248); J. M. Walker (Methods in Molecular Biology (Series), Humana Press, ISSN 1064-3745); Rio et al. (RNA: A Laboratory Manual, 2010, Cold Spring Harbor Laboratory Press, ISBN 978-0879698911); Methods in Enzymology (Series), Academic Press; Green et al. (Molecular Cloning: A Laboratory Manual, 2012, Fourth Edition, Cold Spring Harbor Laboratory Press, ISBN 978-1605500560); and G. T. Hermanson (Bioconjugate Techniques, 2013, Third Edition, Academic Press, ISBN 978-0123822390).

The terms “wild-type,” “naturally occurring,” and “unmodified” are used herein to mean the typical (or most common) form, appearance, phenotype, or strain existing in nature; for example, the typical form of cells, organisms, polynucleotides, proteins, macromolecular complexes, genes, RNAs, DNAs, or genomes as they occur in, and can be isolated from, a source in nature. The wild-type form, appearance, phenotype, or strain serve as the original parent before an intentional modification. Thus, mutant, variant, engineered, recombinant, and modified forms are not wild-type forms.

By “isolated” is meant, when referring to a polypeptide, that the indicated molecule is separate and discrete from the whole organism with which the molecule is found in nature or is present in the substantial absence of other biological macromolecules of the same type. The term “isolated” with respect to a polynucleotide is a nucleic acid molecule devoid, in whole or part, of sequences normally associated with it in nature; or a sequence, as it exists in nature, but having heterologous sequences in association therewith; or a molecule disassociated from the chromosome.

The term “purified” as used herein preferably means at least 75% by weight, more preferably at least 85% by weight, more preferably still at least 95% by weight, and most preferably at least 98% by weight, of the same molecule is present.

The terms “engineered,” “genetically engineered,” “genetically modified,” “recombinant,” “modified,” “non-naturally occurring,” and “non-native” indicate intentional human manipulation of a wild-type nucleic acid or protein. The terms encompass methods of genomic modification that include genomic editing, as defined herein, as well as techniques that alter gene expression or inactivation, enzyme engineering, directed evolution, knowledge-based design, random mutagenesis methods, gene shuffling, codon optimization, and the like. Methods for genetic and protein engineering are known in the art.

“Covalent bond,” “covalently attached,” “covalently bound,” “covalently linked,” “covalently connected,” and “molecular bond” are used interchangeably herein and refer to a chemical bond that involves the sharing of electron pairs between atoms. Examples of covalent bonds include, but are not limited to, phosphodiester bonds and phosphorothioate bonds.

“Non-covalent bond,” “non-covalently attached,” “non-covalently bound,” “non-covalently linked,” “non-covalent interaction,” and “non-covalently connected” are used interchangeably herein, and refer to any relatively weak chemical bond that does not involve sharing of a pair of electrons. Multiple non-covalent bonds often stabilize the conformation of macromolecules and mediate specific interactions between molecules. Examples of non-covalent bonds include, but are not limited to hydrogen bonding, ionic interactions (e.g., Na⁺Cl⁻), van der Waals interactions, and hydrophobic bonds.

As used herein, “sequence identity” generally refers to the percent identity of nucleotide bases or amino acids comparing a first polynucleotide or polypeptide to a second polynucleotide or polypeptide using algorithms having various weighting parameters. Sequence identity between two polynucleotides or two polypeptides can be determined using sequence alignment by various methods and computer programs (e.g., BLAST, CS-BLAST, FASTA, HMMER, L-ALIGN, and the like) available through the worldwide web at sites including, but not limited to, GENBANK (www.ncbi.nlm.nih.gov/genbank/) and EMBL-EBI (www.ebi.ac.uk.). Sequence identity between two polynucleotides or two polypeptide sequences is generally calculated using the standard default parameters of the various methods or computer programs. A high degree of sequence identity between two polynucleotides or two polypeptides is typically between about 90% identity and 100% identity over the length of the reference polypeptide, for example, about 90% identity or higher, preferably about 95% identity or higher, more preferably about 98% identity or higher. A moderate degree of sequence identity between two polynucleotides or two polypeptides is typically between about 80% identity to about 85% identity, for example, about 80% identity or higher, preferably about 85% identity over the length of the reference polypeptide. A low degree of sequence identity between two polynucleotides or two polypeptides is typically between about 50% identity and 75% identity, for example, about 50% identity, preferably about 60% identity, more preferably about 75% identity over the length of the reference polypeptide.

For instance, a sequence of the present disclosure may have a particular sequence identity to a reference sequence. This sequence identity may be, for example, 25% or more, 50% or more, 75% or more, 80% or more, 85% or more, 90% or more, 91% or more, 92% or more, 93% or more, 94% or more, 95% or more, 96% or more, 97% or more, 98% or more, or 99% or more.

As used herein, “binding” refers to a non-covalent interaction between macromolecules (e.g., between a protein and a polynucleotide, between a polynucleotide and a polynucleotide, or between a protein and a protein, and the like). Such non-covalent interaction is also referred to as “associating” or “interacting” (e.g., if a first macromolecule interacts with a second macromolecule, the first macromolecule binds to second macromolecule in a non-covalent manner). Some portions of a binding interaction may be sequence-specific (the terms “sequence-specific binding,” “sequence-specifically bind,” “site-specific binding,” and “site specifically binds” are used interchangeably herein). Binding interactions can be characterized by a dissociation constant (Kd). “Binding affinity” refers to the strength of the binding interaction. An increased binding affinity is correlated with a lower Kd.

A “coding sequence” or a sequence that “encodes” a selected polypeptide, is a nucleic acid molecule that is transcribed (in the case of DNA) and translated (in the case of mRNA) into a polypeptide in vitro or in vivo when placed under the control of appropriate regulatory sequences. The boundaries of the coding sequence are determined by a start codon at the 5′ terminus and a translation stop codon at the 3′ terminus. A transcription termination sequence may be located 3′ to the coding sequence.

As used herein, the term “modulate” refers to a change in the quantity, degree or amount of a function. Thus, “modulation” of gene expression includes both gene activation and gene repression. Modulation can be assayed by determining any characteristic directly or indirectly affected by the expression of the target gene. Such characteristics include, for example, changes in RNA or protein levels, protein activity, product levels, expression of the gene, or activity level of reporter genes.

As used herein, the term “between” is inclusive of end values in a given range (e.g., between 10 and 50 amino acids in length includes 10 amino acids and 50 amino acids).

As used herein, the term “amino acid” refers to natural and synthetic (unnatural) amino acids, including amino acid analogs, modified amino acids, peptidomimetics, glycine, and D or L optical isomers.

As used herein, the terms “peptide,” “polypeptide,” and “protein” are interchangeable and refer to polymers of amino acids. A polypeptide may be of any length. It may be branched or linear, it may be interrupted by non-amino acids, and it may comprise modified amino acids. The terms also refer to an amino acid polymer that has been modified through, for example, acetylation, disulfide bond formation, glycosylation, lipidation, phosphorylation, pegylation, biotinylation, cross-linking, and/or conjugation (e.g., with a labeling component or ligand). Polypeptide sequences are displayed herein in the conventional N-terminal to C-terminal orientation, unless otherwise indicated. Polypeptides and polynucleotides can be made using routine techniques in the field of molecular biology. Furthermore, essentially any polypeptide or polynucleotide is available from commercial sources.

The terms “fusion protein” and “chimeric protein” as used herein refer to a single protein created by joining two or more proteins, protein domains, or protein fragments that do not naturally occur together in a single protein.

A “moiety” as used herein refers to a portion of a molecule. A moiety can be a functional group or describe a portion of a molecule with multiple functional groups (e.g., that share common structural aspects). The terms “moiety” and “functional group” are typically used interchangeably; however, a “functional group” can more specifically refer to a portion of a molecule that comprises some common chemical behavior. “Moiety” is often used as a structural description.

The terms “modified protein,” “mutated protein,” “protein variant,” and “engineering protein” as used herein typically refers to a protein that has been modified such that it comprises a non-native sequence (i.e., the modified protein has a unique sequence compared to an unmodified protein); or a native sequence but with one or more non-native modifications, conjugations, etc.

The terms “subject,” “individual,” or “patient” are used interchangeably herein and refer to any member of the phylum Chordata, including, without limitation, humans and other primates, including non-human primates, such as rhesus macaques, chimpanzees, and other monkey and ape species; farm animals, such as cattle, sheep, pigs, goats, and horses; domestic mammals, such as dogs and cats; laboratory animals, including rabbits, mice, rats, and guinea pigs; birds, including domestic, wild, and game birds, such as chickens, turkeys, and other gallinaceous birds, ducks, and geese; and the like. The term does not denote a particular age or gender. Thus, the term includes adult, young, and newborn individuals as well as males and females.

The terms “effective amount” or “therapeutically effective amount” of a composition or agent, refer to a sufficient amount of the composition or agent to provide the desired response. Preferably, the effective amount will prevent, avoid, or eliminate one or more harmful side-effects. The exact treatment amount required will vary from subject to subject, depending on the species, age, and general condition of the subject, the severity of the condition being treated, and the particular treatment used, mode of administration, and the like. An appropriate “effective” amount in any individual case may be determined by one of ordinary skill in the art using routine experimentation.

“Treatment” or “treating” a particular disease includes: (1) preventing the disease, for example, preventing the development of the disease or causing the disease to occur with less intensity in a subject that may be predisposed to the disease, but does not yet experience or display symptoms of the disease; (2) inhibiting the disease, for example, reducing the rate of development, arresting the development or reversing the disease state; and/or (3) relieving symptoms of the disease, for example, decreasing the number of symptoms experienced by the subject.

As used herein, the term “receptor” refers to a molecule which responds to, and/or binds, specifically to a particular substance or molecule (e.g, a virus, drug, neurotransmitter, hormone, antigen, or other substance).

As used herein, the term “virus” refers to a collection of genetic code, either DNA or RNA, surrounded by a protein coat. The term “coronavirus” or “CoV” refers to viruses belonging to the order Nidovirales, family Coronaviridae, and subfamily Orthocoronavirinae. Coronaviruses are positive-sense RNA viruses that are spherical and enveloped with club-shaped spikes on the surface. Coronaviruses may cause mild (e.g., human coronavirus (HCoV)-229E, HCoV-0C43, HCoV-NL63, and HCoV-HKU1) or more severe disease (e.g., severe acute respiratory syndrome (SARS)-associated coronavirus (SARS-CoV), Middle East respiratory syndrome coronavirus (MERS-CoV), and Severe Acute Respiratory Syndrome Coronavirus 2 (SARS-CoV-2)). SARS-CoV-2 was provisionally known as 2019-nCov. SARS-CoV-2 is a member of subgenus Sarbecovirus of the genus Betacoronavirus and is a positive-sense single-stranded RNA virus that encodes 12 putative open reading frames responsible for ˜27 proteins (NC_045512). The major structural proteins include the spike (S), membrane (M), envelope (E) and the nucleocapsid (N) protein.

“Coronavirus disease 2019 (COVID-19)” refers to the disease caused by SARS-CoV-2.

As used herein, “protein stability,” “thermodynamic stability,” and “stability” are used interchangeably and refer to the net balance of forces which determine whether a protein will be its native folded conformation or a denatured state. Changes in protein stability may be “destabilizing” or “stabilizing.” “Destabilizing” refers to changes or processes that decrease the stability of a protein, making it more vulnerable to degradative processes, denaturation, or aggregation in comparison to the wild-type. “Stabilizing” refers to changes or processes that increase the stability of a protein in comparison to the wild-type.

As used herein the term “binding energy change” refers to “ΔΔΔG” which is the binding energy difference between the wild-type structure and the structure with the ACE2 mutation. The binding energy is the difference between the folding energy of the complex and the individual monomers.

“Gibbs free energy,” “free energy,” and “protein folding free energy” are used interchangeably to refer to the biophysical process of protein folding (ΔG) which represents the thermodynamic stability of a protein. “Folding energy changes” are termed “ΔΔG” and in some embodiments are mutation-induced changes, which are used to estimate the effects of mutations on protein stability.

### Peptides

As described herein, peptides of the present disclosure can be designed and/or developed using bioinformatics and/or computational approaches as discussed herein, such as in the Examples. Peptides can be designed that are derived from, or are part of, a receptor protein for a virus. In some embodiments, the virus is a coronavirus. In certain embodiments thereof, the coronavirus is SARS-CoV-2.

In some embodiments, the receptor protein is a cell surface protein that normally is expressed on the surface of a host cell. In some embodiments, the receptor protein is ACE2, such as human ACE2 (hACE2). In some embodiments, the receptor protein is Transmembrane Serine Protease 2 (TMPRSS2). In some embodiments, TMPRSS2- and ACE2-derived peptides may be used in combination, to synergistically exert inhibitory effects on the infection of SARS-CoV-2.

In some embodiments, peptides of the present disclosure are linear peptides. In other embodiments, peptides may be branched or circular. In some embodiments, peptides of the present disclosure may contain one or more non-naturally-occurring amino acids, and/or one or more modified amino acids. In some embodiments thereof, the modification may include, for example, acetylation, disulfide bond formation, glycosylation, lipidation, phosphorylation, pegylation, biotinylation, cross-linking, and/or conjugation (e.g., with a labeling component or ligand). In some embodiments, the peptide is fused to another peptide domain, to form a fusion protein or chimeric protein.

In some embodiments, the peptide contains less than 100 amino acids. In some embodiments, the peptide contains between 10-50 amino acids. In some embodiments, the peptide contains at least, exactly, or less than, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50, amino acids.

In some embodiments, the peptide has one or more (e.g., at least 1, 2, 3, 4, 5, 6, 7, 8, 9, 10, etc). amino acid additions, deletions, or substitutions, as compared to the corresponding region in the wild-type receptor protein. In some embodiments, a peptide of the present disclosure has 80% or more, 81% or more, 82% or more, 83% or more, 84% or more, 85% or more, 86% or more, 87% or more, 88% or more, 88% or more, 90% or more, 91% or more, 92% or more, 93% or more, 94% or more, 95% or more, 96% or more, 97% or more, 98% or more, or 99% or more, identity to the corresponding region in the wild-type receptor protein.

In some embodiments, the peptide consists of, consists essentially of, or comprises, a peptide sequence selected from STIEEQAKTFLDKFNHEAEDLFYQSSLASWN (SEQ ID NO: 1); NMNNAGDKWSAFLKEQSTLAQMYPLQEIQNLTVKLQLQALQQ (SEQ ID NO: 2); LPNMTQGFWENSML; (SEQ ID NO: 3); AWDLGKGDFRIL (SEQ ID NO: 4); HIQYDMAYAAQPFLLRNG (SEQ ID NO: 5); and QKLFNMLRLGKS (SEQ ID NO: 6). In some embodiments, the sequence and/or the amino acids of any of SEQ ID NOs: 1-6 may be modified, mutated, etc., as discussed herein.

### Delivery of Peptides

Delivery of peptides of the present invention to subjects may be achieved by a number of methods known to one of ordinary skill in the art. Delivery systems may include, for example, aqueous and nonaqueous gels, creams, multiple emulsions, microemulsions, liposomes, ointments, aqueous and nonaqueous solutions, lotions, aerosols, hydrocarbon bases and powders, and can contain excipients such as solubilizers, polymers, etc.

The useful dosage to be administered and the particular mode of administration will vary depending upon such factors as the subject and virus type, including, e.g., the age, weight and the particular animal and region thereof to be treated, the particular agent and delivery method used, the therapeutic use contemplated, and the form of the formulation, for example, suspension, emulsion, etc., as will be readily apparent to those skilled in the art.

A suitable dosage unit of peptide may be in the range of 0.001 to 10 milligrams per kilogram body weight of the recipient per day, or in the range of 0.01 to 1 milligrams per kilogram body weight per day, or in the range of 0.01 to 0.1 milligrams per kilogram body weight per day.

The administered peptides can be used to treat individuals infected with, or suspected of being infected with, a virus, such as a coronavirus, such as SARS-CoV-2.

The administration of the agents and compositions of the present disclosure to subjects may be carried out in any convenient manner, including by aerosol inhalation, injection, ingestion, transfusion, implantation or transplantation. The compositions described herein may be administered to a patient subcutaneously, intradermally, intratumorally, intranodally, intramedullary, intramuscularly, by intravenous or intralymphatic injection, or intraperitoneally. The agents or compositions can be administrated in one or more doses. In some embodiments, an effective amount is administrated as a single dose. In other embodiments, an effective amount is administrated as more than one dose, over a period time. The determination of optimal ranges of effective amounts of a given cell type for a particular disease or condition is within the skill of those in the art.

Treatments of the present disclosure can be provided in conjunction with, before, or after, vaccination, and/or supportive care, such as supplemental oxygen, mechanical ventilation, and steroids.

### ACE2 and Renin-Angiotensin System (RAS) and COVID-19 Comorbidities

ACE2 is a cell membrane protein present in the lungs, arteries, heart, kidneys, and intestines and functions to reduce blood pressure and is anti-hypertensive. See, e.g., Hamming et al. (“Tissue distribution of ACE2 protein, the functional receptor for SARS coronavirus. A first step in understanding SARS pathogenesis,”J. Pathol., 2004, 203: 631-7); Lely et al. (“Renal ACE2 expression in human kidney disease,”J. Pathol., 2004, 204: 587-93); and Donoghue et al. (“A novel angiotensin-converting enzyme-related carboxypeptidase (ACE2) converts angiotensin I to angiotensin 1-9,” Circ. Res., 2002, 87: E1-9). Specifically, ACE2 converts angiotensin II to peptide Ang-(1-7) or angiotensin I to peptide Ang-(1-9); both peptides are vasodilators. See, e.g., Patel et al. (“Recombinant Human ACE2 and the Angiotensin 1-7 Axis as Potential New Therapies for Heart Failure,” Can. J. Cardiol., 2017, 33: 943-946); Patel et al. (“Role of the ACE2/Angiotensin 1-7 Axis of the Renin-Angiotensin System in Heart Failure,” Circ. Res., 2016, 118: 1313-26); Wang et al. (“Angiotensin-Converting Enzyme 2 Metabolizes and Partially Inactivates Pyr-Apelin-13 and Apelin-17: Physiological Effects in the Cardiovascular System,” Hypertension, 2016, 68: 365-77); Keidar et al. (“ACE2 of the heart: From angiotensin I to angiotensin (1-7),” Cardiovasc. Res., 2007, 73: 463-9); and Zisman et al. (“Increased angiotensin-(1-7)-forming activity in failing human heart ventricles: evidence for upregulation of the angiotensin-converting enzyme Homologue ACE2,” Circulation, 2003, 108: 1707-12). ACE2 function is integral to the ACE2 and renin-angiotensin system (RAS), which is necessary for regulating blood pressure, fluid and electrolyte balance, and systemic vascular resistance.

Angiotensin I is generated by renin after being stimulated by several conditions including low blood pressure, bleeding, and dehydration. See, e.g., Patel et al. (“Role of the ACE2/Angiotensin 1-7 Axis of the Renin-Angiotensin System in Heart Failure,” Circ. Res., 2016, 118: 1313-26); Barbosa et al. (“The Renin Angiotensin System and Bipolar Disorder: A Systematic Review,” Protein Pept. Lett., 2020, doi:10.2174/0929866527666200127115059); Sassi et al. (“Renin-angiotensin-aldosterone system and migraine: a systematic review of human studies,” Protein Pept. Lett., 2020, doi:10.2174/0929866527666200129160136); and Zhou et al. (“The renin-angiotensin system blockers and survival in digestive system malignancies: A systematic review and meta-analysis,” Medicine (Baltimore), 2020, 99: e19075). Angiotensin I is then converted to angiotensin II by angiotensin converting enzyme (ACE). See, e.g., Li et al. (“Effects of Angiotensin-Converting Enzyme Inhibitors on Arterial Stiffness: A Systematic Review and Meta-Analysis of Randomized Controlled Trials,” Cardiovasc. Ther., 2020, 7056184); and Ramezani et al. (“Angiotensin-converting enzyme gene insertion/deletion polymorphism and susceptibility to psoriasis: a systematic review and meta-analysis,” BMC Med. Genet., 2020, 21:8). Angiotensin II constricts the vasculature to elevate the blood pressure.

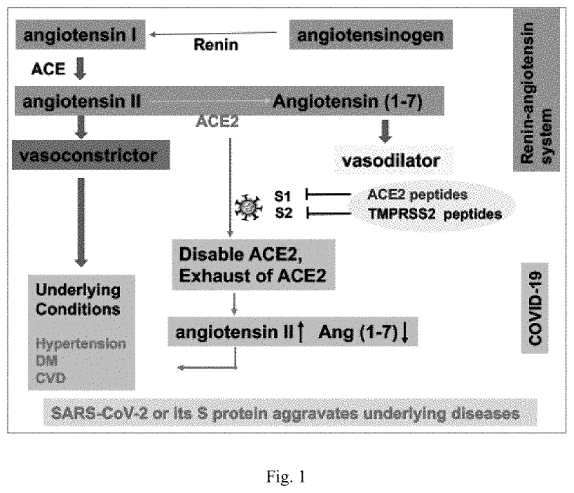

Angiotensin II can be subsequently hydrolyzed by ACE2 to generate angiotensin peptides, mainly Angiotensin-(1-7), also called Ang-(1-7). See, e.g., Keidar et al. (“ACE2 of the heart: From angiotensin I to angiotensin (1-7),” Cardiovasc. Res., 2007, 73: 463-9). Ang-(1-7) is a vascular dilator and thus reduces blood pressure. See FIG. 1. ACE2 also hydrolyzes angiotensin I to form Angiotensin-(1-9) [Ang-(1-9)], another vascular dilator. See, e.g., Delbridge et al. (“Angiotensin-(1-9): New Promise for Post-Infarct Functional Therapy,” J. Am. Coll. Cardiol., 2016, 68: 2667-2669); Fattah et al. (“Gene Therapy With Angiotensin-(1-9) Preserves Left Ventricular Systolic Function After Myocardial Infarction,” J. Am. Coll. Cardiol., 2016, 68:2652-2666); and Sotomayor-Flores et al. (“Angiotensin-(1-9) prevents cardiomyocyte hypertrophy by controlling mitochondrial dynamics via miR-129-3p/PKIA pathway,” Cell Death Differ., 2020, doi:10.1038/s41418-020-0522-3). Abnormally high levels of angiotensin II cause hypertension, aggravate DM sequelae, and induce CVDs, as shown in FIG. 1.

Balanced RAS signaling is necessary for normal blood pressure and cardiovascular function, and is thus vital for an overall healthy cardiovascular system. SARS-CoV-2 infection can aggregative these underlying diseases and lead to increased COVID-19 fatality through, for example, interactions with the S protein.

One theory for explaining how the aforementioned comorbidities significantly increase the fatality of COVID-19 is based on the fact that these patients have a history of taking drugs such as ACE inhibitors (ACEIs), angiotensin II receptor blockers (ARBs), and ibuprofen. See, e.g., Fang et al. (“Antihypertensive drugs and risk of COVID-19?—Authors' reply,” Lancet Respir. Med., 2020, doi:10.1016/S2213-2600(20)30159-4); and Fang et al. (“Are patients with hypertension and diabetes mellitus at increased risk for COVID-19 infection?,” Lancet Respir. Med., 2020, 8:e21). These drugs not only inhibit ACE but also increase the levels of ACE2. The increased levels of ACE2 might result in greater chances of SARS-CoV-2 infection, subsequent viral infection, and ultimately death.

However, this theory does not explain why the onset of COVID-19 in these patients is so rapid, such that emergent rescue is often needed and yet still the disease is fatal. Furthermore, it contradicts the recently published reports that using ARBs and ACEIs improved COVID-19. See, e.g., Meng et al. (“Renin-angiotensin system inhibitors improve the clinical outcomes of COVID-19 patients with hypertension,” Emerg. Microbes Infect., 2020, 9: 757-760); Acanfora et al. (“Neprilysin inhibitor-angiotensin II receptor blocker combination (sacubitril/valsartan): rationale for adoption in SARS-CoV-2 patients,” Eur. Heart J. Cardiovasc. Pharmacother., 2020, doi:10.1093/ehjcvp/pvaa028); and Gurwitz et al. (“Angiotensin receptor blockers as tentative SARS-CoV-2 therapeutics,” Drug Dev. Res., 2020, doi:10.1002/ddr.21656). Further, a critical review analyzed the available evidence and found that it does not support a deleterious effect of RAS blockers in COVID-19. See, e.g., Kreutz et al. (“Hypertension, the renin-angiotensin system, and the risk of lower respiratory tract infections and lung injury: implications for COVID-19,” Cardiovasc. Res., 2020, doi:10.1093/cvr/cvaa097).

Nonetheless, the released free spike protein or the cleaved S1 protein circulating in blood may bind to ACE2 present in the heart, arteries, and alveolar lung cells, ultimately blocking the conversion of Angiotensin II to Ang-(1-7) and/or Angiotensin I to Ang-(1-9); this is supported by a previous experiment on SARS-CoV-1. See Kuba et al. (“A crucial role of angiotensin converting enzyme 2 (ACE2) in SARS coronavirus-induced lung injury,” Nat. Med., 2005, 11: 875-9).

Thus, the interaction of ACE2 and the SARS-CoV-2 S protein might exhaust ACE2 or compromise ACE2 function to exacerbate underlying diseases and increase COVID-19-related morbidity and mortality. As discussed herein, novel peptides which target the S protein to inhibit viral infection and preserve physiological functions of ACE2 may be used to improve conditions of COVID-19 patients.

### EXAMPLES

Non-limiting embodiments of the present invention are illustrated in the following Examples. Efforts have been made to ensure accuracy with respect to numbers used (e.g., amounts, concentrations, percent changes, and the like), but some experimental errors and deviations should be accounted for.

Unless indicated otherwise, temperature is in degrees Centigrade and pressure is at or near atmospheric. It should be understood that these Examples are given by way of illustration only and are not intended to limit the scope of what the inventor regards as various embodiments of the present invention. Not all of the following steps set forth in each Example are required nor must the order of the steps in each Example be as presented.

### Computational Structure-Based ACE2- and TMPRSS2-Derived Peptides

Stable and effective therapeutic peptides for the treatment of COVID-19 were designed and developed using a novel computational structure-based energy calculation approach. The present inventors developed several ACE2-derived peptides which strongly interact with S1. The present inventors surmised that these novel peptides targeting the S protein, specifically S1, could treat SARS-CoV-2 infection by 1) reducing the interaction of S1 with ACE2, so that the function of ACE2 will not be affected; and 2) binding to viral particles to block viral infection. Similarly, the present inventors surmised that peptides derived from TMPRSS2 may be capable of inhibiting viral infection by binding the S2 protein on viral particles. See FIG. 1. Similar bioinformatics approaches as described herein for ACE2 may be used to identify TMPRSS2-derived peptides that will bind S2.

In developing these peptides (SEQ ID NOs: 1-6), the features, including stability and binding affinity to the S protein, of human angiotensin converting enzyme 2 (hACE2) and human transmembrane protease, serine 2 (hTMPRSS2), were analyzed. Bioinformatics approaches were used to quantitatively assess the effects of disease-causing mutations on protein stability and protein-protein interactions. Computational saturation mutagenesis approaches, which are an important alternative to experimental mutagenesis approaches, were then used to quantify the systemic effects of coding mutations on the receptor-binding domain (RBD)-ACE2 interaction.

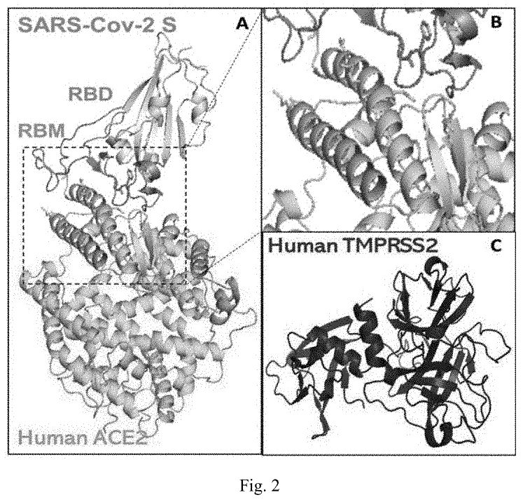

Recent structure determinations revealed the atomic details of the SARS-CoV-2 S protein and the binding interface between RBD and hACE2. See Teng et al. (“Systemic Effects of Missense Mutations on SARS-CoV-2 Spike Glycoprotein Stability and Receptor Binding Affinity,” Brief Bioinform., 2021, 22(2): 1239-1253). As shown in FIGS. 2A and 2B, the RBD of SARS-CoV-2 S protein includes a core and a receptor-binding motif (RBM) that specifically recognizes the interacting regions of hACE2. The structure of the RBD of the S protein complexed with the hACE2 receptor (ID: 6LZG) was obtained from Protein Data Bank (PDB), and the effects of hACE2 mutations on the RBD-hACE2 binding affinity and hACE2 protein stability were determined. ACE2-derived peptides were designed using the binding energy changes upon hACE2 mutations. The binding energy is the difference between the folding energy of the complex and the individual monomers. The binding energy change is calculated as the binding energy difference between the wild-type structure and the structure with the ACE2 mutation. SWISS-MODEL, see Biasini et al. (“SWISS-MODEL: modelling protein tertiary and quaternary structure using evolutionary information,” Nucleic Acids Res., 2014, 42: W252-8), was used to create the structure of hTMPRSS2. See FIG. 2C).

### Human ACE2 Mutations Affecting ACE2 Stability

As discussed herein, ACE2 is the receptor for S1 binding and is critical in initiating SARS-CoV-2 infection. ACE2 also determines the permissiveness of cells for SARS-CoV-2 infection. While, due to structural and functional studies, there is a significant understanding of ACE2, the stability of ACE2 has not been scrutinized. The present inventors determined the effects of 11,324 mutations generated from 596 residues from the hACE2 chain of SARS-CoV-2 S RBD-ACE2 complex on destabilization and stabilization of hACE2.

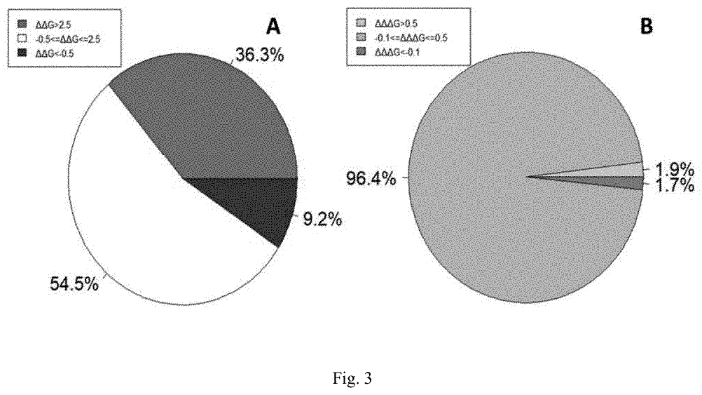

As shown in FIG. 3A, of the 11,324 mutations assessed, 4,105 (36.3%) mutations have a destabilizing effect (ΔΔG>2.5 kcal/mol) on hACE2 protein, 1,044 (9.3%) mutations have a stabilizing effect (ΔΔG<−0.5 kcal/mol) on hACE2 protein, and the remaining 6,175 (54.5%) have no effects (−0.5<=ΔΔG<=2.5 kcal/mol) on hACE2 stability.

As shown in TABLE 1, the mutations at hACE2 residue positions G561, G486, G268, G405, G399 induce highly destabilizing effects on hACE2 protein stability. The maximum average change of ΔΔG of residues is G561 at 26.105 kcal/mol. Mutation G561W has the first-largest folding energy change (ΔΔG) at 53.578 kcal/mol, and G561F has the fourth-largest ΔΔG at 50.099 kcal/mol. Residue G486 has the second-largest mean ΔΔG at 25.205, with mutations G486F (ΔΔG=52.817 kcal/mol) and G486H (ΔΔG=49.409 kcal/mol), and can also significantly reduce hACE2 stability.

TABLE 1 depicts the effects of hACE2 key residues and gnomAD variations on protein stability.

| | | | | | | | gnomAD |
|---|---|---|---|---|---|---|---|
| | | | | | | | Allele |
| Residues | Mean | Mutation | ΔΔΔG | dbSNP151 | Mutation | ΔΔΔG | Count |
| G561 | 26.105 | G561W | 53.578 | rs4646116 | K26R | 0.461 | 728 |
| G486 | 25.205 | G486F | 52.817 | rs148771870 | G211R | 1.041 | 242 |
| G268 | 18.049 | A403W | 51.244 | rs191860450 | I468V | 1.345 | 154 |
| G405 | 17.897 | G561F | 50.099 | rs138390800 | K341R | −0.079 | 59 |
| G399 | 15.574 | G486H | 49.409 | rs372272603 | R219C | 1.928 | 59 |
| D350 | −1.6 | E375L | −4.506 | rs771621249 | N58K | −1.071 | 2 |
| D382 | −1.131 | E375I | −4.071 | rs763395248 | T92I | −1.032 | 2 |
| G66 | −0.968 | Q526F | −3.993 | rs142984500 | H378R | 7.026 | 15 |
| S105 | −0.882 | D350F | −3.776 | rs767462182 | G377E | 11.772 | 1 |
| S47 | −0.843 | E375V | −3.748 | rs763655186 | G448E | 17.293 | 1 |

The ACE2 residue position D350 has the lowest ΔΔG mean value at −1.6 kcal/mol. Mutation D350F (ΔΔG=−3.776 kcal/mol) in this position can make the hACE2 receptor stable. Other residues stabilizing hACE2 include D382, G66, S105, and S47. Mutation E375L has the largest negative folding free energy change at −4.506 kcal/mol, E375I and E375V in this position also have highly stabilizing effects on hACE2 protein stability.

The folding energy change caused by human variations present in the Genome Aggregation Database (gnomAD) were also investigated. Notably, the common variants, such as K26R with 728 allele counts and G211R with 242 allele counts, have moderate destabilizing effects on hACE2 stability. Mutations N58K and T92I can increase the protein stability of hACE2. In contrast, mutations G448E, G377E, and H378R can destaiblize the hACE2 protein significantly.

### Effects of hACE2 Mutations on RBD-hACE2 Interaction

The effects of the 11,324 total mutations on the hACE2 chain of the crystal structure of the RBD-hACE2 complex were assessed to determine whether the human ACE2 mutations located in RBD-hACE2 interface affected the interaction of hACE2 with the RBD of the SARS-Cov-2 S protein. Of the 11,324 mutations assessed, 10,921 (96.4%) hACE2 mutations had no effect (−0.1<=ΔΔΔG<0.1 kcal/mol) on RBD-ACE2 binding affinity (FIG. 3B). Whereas, 210 (1.9%) mutations were determined to decrease (ΔΔΔG>0.5 kcal/mol) the binding affinity of the RBD-hACE2 complex, and in contrast, 193 mutations (1.7%) with ΔΔΔG <-0.1 kcal/mol, were determined to increase the binding affinity of the RBD-hACE2 complex.

As shown in TABLE 2, the mutations at residues D355, D38, and Q42 induce strong destabilizing effects on RBD-hACE2 binding affinity. Residue D355 (ΔΔΔG mean=2.031 kcal/mol) and mutation D355Y (ΔΔΔG=7.284 kcal/mol) have maximum effects on destabilizing the RBD-hACE2 complex. Whereas, the mutations at residues Y41, K353, and N330 can increase the RBD-hACE2 binding affinity. Residue Y41 (ΔΔΔG mean=−0.742 kcal/mol) and mutation Y41P (ΔΔΔG=−1.808 kcal/mol) can also increase the RBD-hACE2 interaction. K353F has the smallest binding free energy change at −1.937 kcal/mol, and K353C and K353Y can also increase the binding affinity of the RBD-hACE2 complex.

TABLE 2 depicts the effects of hACE2 key residues and gnomAD variations on RBD-ACE 2 interaction.

| | | | | | | | gnomAD |
|---|---|---|---|---|---|---|---|
| | | | | | | | Allele |
| Residues | Mean | Mutation | ΔΔΔG | dbSNP151 | Mutation | ΔΔΔG | Count |
| D355 | 2.031 | D355Y | 7.284 | rs4646116 | K26R | 0.021 | 728 |
| D38 | 1.858 | G326Y | 6.967 | rs148771870 | G211R | 0.000 | 242 |
| Q42 | 1.392 | D355W | 6.681 | rs191860450 | I468V | 0.000 | 154 |
| G354 | 1.298 | D355R | 4.139 | rs138390800 | K341R | 0.000 | 59 |
| M82 | 1.19 | K31P | 4.036 | rs1299103394 | K26E | −0.216 | 1 |
| Y41 | −0.742 | K353F | −1.937 | rs370610075 | G352V | −0.203 | 1 |
| K353 | −0.47 | K353C | −1.870 | rs143936283 | E329G | −0.078 | 5 |
| N330 | −0.44 | T27F | −1.839 | rs961360700 | D355N | 1.338 | 2 |
| L351 | −0.172 | Y41P | −1.808 | rs73635825 | S19P | 0.962 | 46 |
| E329 | −0.154 | K353Y | −1.676 | rs1348114695 | E35K | 0.854 | 3 |

The binding energy change induced by human variations was also investigated. The most common variant, K26R with 728 allele counts, had no effect on RBD-hACE2 binding affinity. Whereas mutations G352V and E329G can enhance the RBD-hACE2 interaction. However, mutations D355N, S19P, and E35K can make the complex unstable.

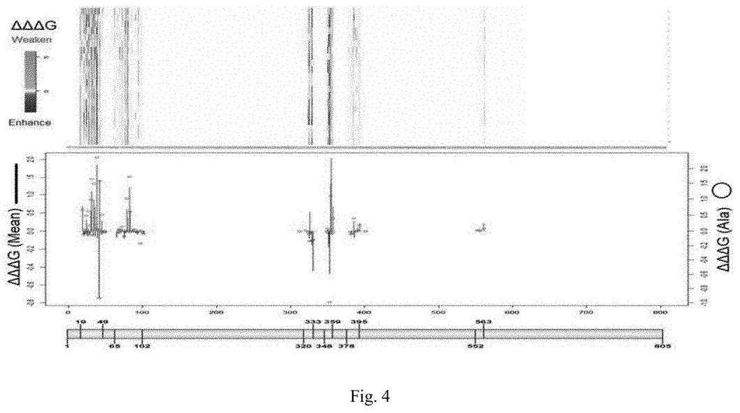

Importantly, FIG. 4 shows that six hACE2 regions (protein positions 19-49, 65-102, 320-333, 348-359, 378-395, 552-563) can interact with SARS-CoV-2 S protein RBD. These six regions include the mutations that have significant effects on RBD-hACE2 binding affinity (|ΔΔΔG|>0.01 kcal/mol). The residues with significant effects on binding affinity are located in these regions. For example, Y41, D38, and Q42 are in the 1st region (19-49). The 4th region (348-359) includes the key residues such as D355 and K353. These six regions are in the binding interfaces of RBD-ACE complex and are involved in the interaction network (FIGS. 3A and 3B). ACE2-derived peptides were designed based on these interacting regions and their functions in preventing infection was validated.

### Mutation Pathogenicity of SARS-CoV-2 S Protein and ACE2

Protein destabilization is a common mechanism by which mutations cause human diseases. The effect of a mutation on protein function can be related to its impact on changes in protein stability. See, e.g., Bromberg et al. (“Correlating protein function and stability through the analysis of single amino acid substitutions,” BMC Bioinformatics, 2009, 10 Suppl 8:S8). Studies suggest that up to 80% of the disease-causing missense mutations may lead to protein destabilization. See, e.g., Wang et al. (“SNPs, protein structure, and disease,” Hum. Mutat., 2001, 17: 263-70). A sequence-based bioinformatics tool, SNAP (screening for non-acceptable polymorphisms), see Bromberg et al. (“SNAP predicts effect of mutations on protein function,” Bioinformatics, 2008, 24: 2397-8), was used to analyze the mutation pathogenicity of all mutations in hACE2.

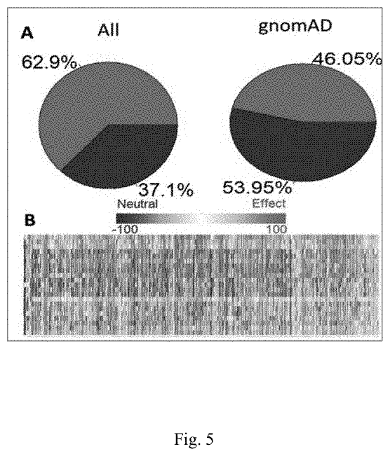

As shown in FIG. 5A, of 15,295 human ACE2 mutations, 9,628 (62.9%) mutations have damaging effects on hACE2 function and 5,667 (37.05%) mutations are predicted as neutral variations. The heatmap of hACE2 also suggests that most of the mutations have damaging effects on hACE2 function (FIG. 5B). Interestingly, more than half of the human variations (53.95%) from gnomAD have neutral effects on hACE2 function and 46.5% of human variations are predicted as deleterious mutations. This finding implies that the human genetics variations tend to occur in the mutations with neutral effects on protein function under the selection pressure.

This data support a novel system for studying the effects of peptides as antivirals and demonstrates that computationally identified peptides are able to attenuate the viral infection in hACE2-complemented cells. Thus, such ACE2-derived peptides may also be useful for other viruses that utilize ACE-RBD for entry. Further, targeting the viral entry mechanisms may be effective in other highly pathogenic and low pathogenic coronaviruses.

### ACE2-Derived Peptides Significantly Attenuated SARS-CoV-2 Infection

The effects of ACE2-derived peptides on infection of SARS-CoV-2 were assessed in a cell culture system, specifically a pseudotyped viral particle system. Using a pseudotyped viral particle system circumvents the need for a BSL-3 facility. The Pseudotyped ΔG-GFP (G*ΔG-GFP) rVSVw/pCAGGS-G-Kan system was obtained from Kerafast Company (www.kerafast.com/productgroup/171/pseudotyped-g-gfp-gg-gfp-rvsv). ΔG-GFP is a replication-restricted, recombinant vesicular stomatitis virus (rVSV) that can be used to produce pseudotyped viruses containing the envelope glycoproteins from a wide variety of heterologous viruses, including those that require BSL-3 or BSL-4 biocontainment; however, because the infectivity of rVSV-ΔG pseudotypes is restricted to a single round of replication, the pseudotypes can be handled using BSL-2 containment practices. These properties, together with the rapid replication kinetics of rVSV-ΔG pseudotypes, have proven useful in studies designed to identify cellular receptors for numerous viruses, and they also provide a robust platform to screen libraries for entry inhibitors and to evaluate the neutralizing antibody responses following vaccination. Such a pseudovirion has been demonstrated to successfully infect hACE2-expressing cells. See, e.g., Ou et al. (“Characterization of spike glycoprotein of SARS-CoV-2 on virus entry and its immune cross-reactivity with SARS-CoV,” Nat. Commun., 2020, 11: 1620).

Briefly, the spike gene of Wuhan-Hu-1 strain (GenBank: MN908947) was codon-optimized for expression in human cells and cloned into the eukaryotic expression plasmid pCAG to generate pCAG-nCoV-S. Plasmid pCAG-nCoV-S was transfected into Vero-E6 for 48 hours, the VSVdG-EGFP-G virus, see Whitt et al. (“Generation of VSV pseudotypes using recombinant DeltaG-VSV for studies on virus entry, identification of entry inhibitors, and immune responses to vaccines,”J. Virol. Methods, 2010, 169: 365-74), was inoculated to the cell expressing SARS-CoV-2 spike protein for 1 hour. Then VSVdG-EGFP-G virus-containing supernatant was replaced with medium containing anti-VSV-G rat serum that blocks the infection of residual rVSVdG-G. The supernatant was harvested at 24 hours post rVSVdG-G infection and centrifuged and filtered (0.45-μm pore size) to remove cell debris, aliquoted and stored at −80° C. for use. Gradient 2× diluted synthetic peptides (ACE2-derived peptides) from 250 nM to 1.95 nM were mixed with VSV-SARS-CoV-2-S virus (MOI=0.05) and incubated at 37° C. for 1 hour. The mixtures were added to BHK21-hACE2 cell for 12 hours. Fluorescent assays were performed.

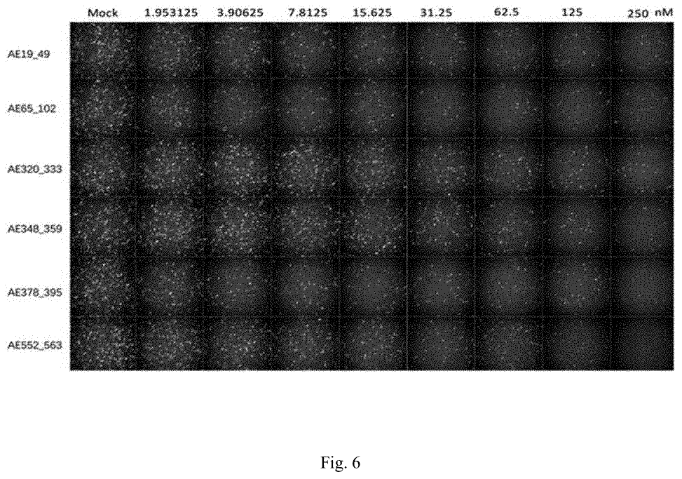

As shown in FIG. 6, all six peptides derived from hACE2 significantly attenuated the infection of the Pseudotyped SARS-CoV-2 infection. Inhibitory effects are apparently associated with the peptide concentrations.

While the subject matter disclosed herein has been described in connection with what is presently considered to be practical example embodiments, it is to be understood that the present disclosure is not limited to the disclosed embodiments, and covers various modifications and equivalent arrangements included within the spirit and scope of the appended claims.

What is claimed is:

1. A composition for the treatment of a viral infection, wherein said composition comprises a first peptide,
wherein said first peptide is derived from a wild-type receptor for the virus, and
wherein said first peptide is able to bind to said virus.

2. The composition of claim 1, wherein said composition is formulated for intranasal administration.

3. The composition of claim 1 or 2, wherein said virus is a coronavirus.

4. The composition of claim 3, wherein said coronavirus is SARS-CoV-2.

5. The composition of claim 4, wherein said first peptide binds to the RBD of a spike protein of the SARS-CoV-2 viral particle.

6. The composition of claim 4, wherein said receptor is ACE2.

7. The composition of claim 4, wherein said receptor is TMPRSS2.

8. The composition of claim 5, wherein said first peptide comprises an amino acid sequence having at least 90% identity to an amino acid sequence selected from the group consisting of SEQ ID NOs: 1-6, and wherein said first peptide is less than 100 amino acids in length.

9. The composition of claim 5, wherein said first peptide comprises an amino acid sequence selected from the group consisting of SEQ ID NOs: 1-6, and wherein said first peptide is less than 100 amino acids in length.

10. The composition of claim 5, wherein said first peptide consists of an amino acid sequence selected from the group consisting of SEQ ID NOs: 1-6.

11. The composition of any one of claims 1-7, wherein said first peptide is 10-50 amino acids in length.

12. The composition of any one of claims 1-11, wherein said composition contains at least one additional peptide derived from a wild-type receptor for the virus,
wherein said additional peptide is able to bind to said virus, and
wherein said at least one additional peptide has a different amino acid sequence from said first peptide.

13. The composition of claim 12, wherein said at least one additional peptide comprises an amino acid sequence having at least 90% identity to an amino acid sequence selected from the group consisting of SEQ ID NOs: 1-6, and wherein said at least one additional peptide is less than 100 amino acids in length.

14. The composition of claim 12, wherein said at least one additional peptide comprises an amino acid sequence selected from the group consisting of SEQ ID NOs: 1-6, and wherein said at least one additional peptide is less than 100 amino acids in length.

15. The composition of claim 12, wherein said at least one additional peptide consists of an amino acid sequence selected from the group consisting of SEQ ID NOs: 1-6.

16. A method of treating a viral infection, comprising administering a therapeutically effective amount of the composition of any one of claims 1-15 to a subject in need thereof.

17. The method of claim 16, wherein said composition is administered intranasally to a subject in need thereof.

18. The method of claim 16 or 17, wherein said first peptide binds to a SARS-CoV-2 viral particle.

19. The method of claim 18, wherein said binding inhibits attachment of said SARS-CoV-2 viral particle to an ACE2 receptor on the surface of a cell of said subject.

20. The method of claim 19, wherein said administration reduces COVID-19-related morbidity and/or mortality in a patient infected with SARS-CoV-2.

21. A method of producing a viral receptor-derived peptide, said method comprising quantifying an effect of a coding mutation on receptor stability and/or receptor-virus binding affinity,
wherein said peptide is derived from a region of said viral receptor that includes the residue subject to said coding mutation.

22. The method of claim 21, wherein the effect of said coding mutation is quantified using computational mutagenesis.

23. The method of claim 21, wherein the peptide is chemically synthesized.

24. The method of claim 21, wherein the peptide is produced by recombinant protein expression.

25. The method of claim 21, wherein said viral receptor is a human protein.

26. The method of claim 21, wherein a binding energy change is used to determine the effect of said coding mutation on receptor stability and/or receptor-virus binding affinity.

27. A method for the treatment of a coronavirus infection in a patient who has an underlying comorbidity consisting of being 65 years or older, having hypertension, having diabetes mellitus, and/or having cardiovascular disease, wherein said method comprises administering a therapeutically effective amount of the composition of any one of claims 4-10 and 13-15 to a subject in need thereof.

28. The composition of claim 12, wherein said first peptide is an ACE2-derived peptide, and wherein said at least one additional peptide is a TMPRSS2-derived peptide.

29. A method of treating a viral infection, comprising administering a therapeutically effective amount of the composition of claim 28 to a subject in need thereof, and wherein said administration has an additive or synergistic effect on treating the viral infection.


SEQUENCE LISTING (6 sequences)

SEQ ID NO: 1 - protein, 31 aa, synthetic construct
STIEEQAKTFLDKFNHEAEDLFYQSSLASWN

SEQ ID NO: 2 - protein, 42 aa, synthetic construct
NMNNAGDKWSAFLKEQSTLAQMYPLQEIQNLTVKLQLQALQQ

SEQ ID NO: 3 - protein, 14 aa, synthetic construct
LPNMTQGFWENSML

SEQ ID NO: 4 - protein, 12 aa, synthetic construct
AWDLGKGDFRIL

SEQ ID NO: 5 - protein, 18 aa, synthetic construct
HIQYDMAYAAQPFLLRNG

SEQ ID NO: 6 - protein, 12 aa, synthetic construct
QKLFNMLRLGKS